(19) United States
(12) Patent Application Publication
(10) Pub. No.: US 2005/0020646 A1
(43) Pub. Date: Jan. 27, 2005

(54) Treatment of BPH

(75) Inventors: Donald Thomas Newgreen, Sandwich (GB); Karl-Erik Andersson, Sandwich (GB); Annette Schroder, Sandwich (GB)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/894,724

(22) Filed: Jul. 19, 2004

Related U.S. Application Data
(60) Provisional application No. 60/496,771, filed on Aug. 21, 2003.

(30) Foreign Application Priority Data
Jun. 25, 2003 (GB) 0317500.7

(51) Int. Cl. A61K 31/433; A61K 31/4245; A61K 31/427; A61K 31/422; A61K 31/416; A61K 31/4025

(52) U.S. Cl. 514/364; 600/362; 514/365; 514/372; 514/374; 514/383; 514/406; 514/422; 514/381; 514/444

(57) ABSTRACT

The present invention relates to the use of EP1 receptor antagonists for the treatment of lower urinary tract symptoms (LUTS) associated with benign prostatic hyperplasia (BPH). The invention also includes screening methods to identify compounds useful for the treatment of LUTS associated with BPH.

This application claims priority to U.S. Provisional Application Ser. No. 60/496,771, filed Aug. 21, 2003, which claims priority to GB Application Serial No. 0317500.7, filed Jul. 25, 2003.

### FIELD OF THE INVENTION

The present invention relates to the use of EP1 receptor antagonists for the treatment of lower urinary tract symptoms (LUTS) associated with benign prostatic hyperplasia (BPH).

The present invention also relates to a method of treatment of LUTS associated with BPH.

The present invention also relates to assays to screen for compounds useful in the treatment of LUTS associated with BPH.

### INTRODUCTION

BPH is a disease which results in the appearance of a characteristic spectrum of lower urinary tract symptoms (LUTS) which comprise ‘voiding’ symptoms directly due to the outflow obstruction such as reduced urinary flow or hesitancy during voiding and ‘storage’ symptoms due to secondary effects on the bladder which include increased day-time and night-time urinary frequency and urgency. Emerging clinical evidence suggests that the ‘storage’ symptoms of BPH, particularly increased night-time frequency (nocturia) are the most bothersome to the patients and result in a greater reduction in quality of life than the ‘voiding’ symptoms. Thus a drug which relieves the ‘storage’ symptoms of BPH either alone or in combination with a drug which targets the ‘voiding’ symptoms e.g. an alpha adrenergic receptor antagonist would be expected to confer a therapeutic benefit.

BPH patients suffer from increased urinary frequency and urgency—a strong desire to void. Urodynamic investigations in BPH patients demonstrate the presence of unstable or non-voiding contractions during bladder filling. The unstable contractions are believed to underlie some or all of the bladder symptoms associated with BPH.

Prostanoids are endogenous signaling molecules, produced locally at their site of action via the enzymatic transformation of arachidonic acid by cyclooxygenase (COX) isoenzymes. Five naturally occurring prostanoids (PGD₂, PGE₂, PGF_{2α}, PGl₂ and TXA₂) have been identified together with corresponding receptor families (DP, EP, FP, IP and TP) through which they mediate their actions (Narumiya et al (1999) Physiol. Rev. 79, 1193-1226).

Prostanoid synthesis occurs locally in both bladder smooth muscle and mucosa, and is initiated by various physiological stimuli, such as stretch of the detrusor muscle and nerve stimulation, and also by injuries and mediators of inflammation (Maggi, C. A. (1992) Pharmacol Res. 25, 13). Biopsies taken from human bladder have shown all five prostanoids to be present (Jeremy et al. (1987) Br. J. Urol. 59, 36-9; Palea et al. (1998) Br. J. Pharmacol. 124, 865-872). However, the contribution of prostanoids to normal physiological or indeed pathophysiological bladder function has not been well defined. Furthermore the prostanoid receptor families and subtypes thereof which mediate either physiological or pathophysiological actions of prostanoids on bladder function have not been characterised.

However, prostaglandin E2 (PGE₂) has been implicated as an endogenous modulator of bladder function, both in the normal physiological state and under pathophysiological conditions (Maggi, C. A. (1992) Pharmacol Res. 25, 13).

In a human volunteer study, intravesical administration of prostaglandin PGE₂ decreased bladder capacity and gave rise to sensations of urgency (Schussler (1990) Urol. Res. 18, 349-352). Topical adminstration of PGE₂ in rats results in bladder hyperactivity and stimulation of reflex micturition (Ishizuka, O. et al (1995) J Urol. 153, 2034). Furthermore in a rat spinal cord injury model of overactive bladder, an increase in the release of PGE₂ from the bladder has been demonstrated.

PGE₂ produces its endogenous activity via the EP-receptor family of G protein coupled receptors, of which 4 subtypes are known to date (Narumiya et al (1999) Physiol. Rev. 79, 1193-1226). The receptor subtype(s) which mediate the actions of PGE₂ on bladder function have not been characterised. However, evidence for a role of the EP1 receptor is provided by studies carried out using EP1 selective antagonists. Thus SC-19220 increased bladder capacity in normal rats (Maggi et al. (1988) Eur. J. Pharmacol. 152, 273-279), and in a rat spinal cord injury model of overactive bladder, the EP1 antagonist ONO-8711 demonstrated a reduction in bladder overactivity (Yoshida et al. (2000) AUA abstracts).

Experimental models of BPH involving bladder outflow obstruction have been developed in a number of animal species. These models which involve the placement of a ligature or disc around the urethra, mimic prostatic occlusion of the urethra and result in the appearance of non-voiding or unstable contractions of the bladder on cystometrical evaluation (Levin et al. (2000) In: Prostatic Diseases (eds Lepor and Oesterling), W B Saunders & Co.). Increased voiding frequency is also a feature of bladder outflow obstruction models thus mimicking the key BPH symptom of increased urinary frequency. The expression of cyclooxygenase-2 (COX-2) has been reported to be increased as a consequence of bladder outflow obstruction in rats (Park, J. M. et al. (1999) Am J. Physiol. 276: F129; Park, J. M. et al (1997) Am J. Physiol. 273: F538), suggesting a possible role of prostanoids in the resulting bladder hyperactivity. However, the identity of any prostanoids involved in bladder dysfunction secondary to outflow obstruction or indeed the receptor families or subtypes which mediate any actions of prostanoids have not been reported.

A mouse model of short term urethral obstruction has been characterised and demonstrated to show increased voiding frequency and the presence of non-voiding contractions, coupled with a reduced bladder capacity (Schroder et al. (2003) J.Urol. 170, 1017-1021). The advantage of this model is that it closely mimics the bladder dysfunction observed in BPH patients and can be applied to transgenic mice. This model has been used to investigate the role of the EP1 receptor in the development of bladder dysfunction following outflow obstruction.

### ASPECTS OF THE INVENTION

A seminal finding of the present invention is the ability to treat the lower urinary tract symptoms (LUTS) associated with BPH with an antagonist for EP1 receptors.

Therefore the invention relates to EP1 receptor antagonists for use in the treatment of the lower urinary tract symptoms (LUTS) associated with BPH. The invention also relates to the use of EP1 receptor antagonists for the manufacture of a medicament for the treatment of LUTS associated with BPH. The invention also relates to a method of treatment of LUTS associated with BPH with an antagonist to EP1 receptors. One aspect of the invention is therefore a method of treating LUTS associated with BPH, comprising the administration to a patient in need of such treatment of an effective amount of an EP1 receptor antagonist. The term “the lower urinary tract symptoms (LUTS) associated with BPH” includes increased day-time and/or night-time frequency, episodes of urgency (strong desire to void) and involuntary loss of urine. Increased nighttime frequency is also known as nocturia. The term “treating the lower urinary tract symptoms (LUTS) associated with BPH” includes the palliative, curative and prophylactic treatment of the lower urinary tract symptoms (LUTS) associated with BPH, complications arising from LUTS associated with BPH and other associated conditions, including increased day-time and/or night-time frequency, episodes of urgency (strong desire to void) and involuntary loss of urine.

The EP1 receptor antagonists preferably will have an IC₅₀ in a ligand binding assay of less than 100 nM, more preferably an IC₅₀ of less than 10 nM, even more preferably an IC₅₀ of less than 1 nM. The IC₅₀ may be measured in a ligand binding assay, e.g. as described in Example 2, or in a functional assay measuring, for example, an increase in intracellular calcium (see, for example, Example 3).

Preferably the EP1 receptor antagonists will be at least 10 fold selective over the EP2 receptor, more preferably at least 100 fold selective over the EP2 receptor. Preferably the EP1 receptor antagonists will be at least 10 fold selective over the EP3 receptor, more preferably at least 100 fold selective over the EP3 receptor. Preferably the EP1 receptor antagonists will be at least 10 fold selective over the EP4 receptor, more preferably at least 100 fold selective over the EP4 receptor. More preferably, the EP1 receptor antagonists will be at least 10 fold selective over the EP2 receptor and at least 10 fold selective over the EP3 and at least 10-fold selective over the EP4 receptor; most preferably at least 100 fold selective over the EP2 receptor and at least 100 fold selective over the EP3 and at least 100-fold selective over the EP4 receptor.

Suitable EP1 receptor antagonists include, for example, SC-51322 (Hallinan et al (1994) Bioorg Med Chem Lett 4, 509-514), SC-19220 and analogues thereof as discussed in Hallinan et al ((1993) J.Med.Chem. 36, 3293-3299), or SC-51089 and analogues thereof as described in Hallinan et al ((1996) J.Med.Chem. 39, 609-613), ZD-4953 and analogues thereof (Ruel et al, Bioorg Med Chem Lett 9, 2699-2704), ONO-8711 (EP 878465), ONO-8713 (WO 00/69465), and others.

Suitable receptor antagonists may also be found in patent applications WO 03/043655, EP 878465, WO 02/072564, WO 02/072098, WO 02/072145, WO 00/69465, or WO 97/00863.

Yet further suitable EP1 receptor antagonists are compounds as described in WO 03/084917. These compounds include compounds of formula (I): 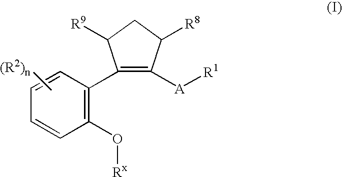
wherein:
A represents an optionally substituted phenyl, or an optionally substituted 5- or 6-membered heterocyclyl ring, or an optionally substituted bicyclic heterocyclyl group;
R¹ represents CO₂R⁴, CONR⁵R⁶, CH₂CO₂R₄, optionally substituted alkyl, optionally substituted alkenyl, optionally substituted SO₂alkyl, SO₂NR⁵R⁶, NR⁵CONR⁵R⁶, CONR⁵R⁶, 2H-tetrazol-5-yl-methyl or optionally substituted heterocyclyl;
R² independently represents halo, optionally substituted alkyl, CN, SO₂R⁵, SR⁵, NO₂, optionally substituted aryl, CONR⁵R⁶ or optionally substituted heteroaryl;
R^{x} represents optionally substituted alkyl wherein 1 or 2 of the non-terminal carbon atoms may optionally be replaced by a group independently selected from NR⁴, O or SOₙ,
wherein n is 0, 1 or 2: or R^{x} may be optionally substituted CQ₂-heterocyclyl or optionally substituted CQ₂-phenyl wherein Q is independently selected from hydrogen and CH₃;
R⁴ represents hydrogen or an optionally substituted alkyl;
R⁵ represents hydrogen or an optionally substituted alkyl;
R⁶ represents hydrogen or an optionally substituted alkyl, optionally substituted SO₂aryl, optionally substituted SO₂heterocyclyl group, CN, optionally substituted CH₂aryl or COR⁷;
R⁷ represents hydrogen, optionally substituted heteroaryl or optionally substituted aryl;
R⁸ and R⁹ independently represent hydrogen or alkyl; and
n is an integer from 0 to 2;
wherein when A is a 6-membered ring the R¹ and cyclopentene group are attached to carbon atoms 1,2-, 1,3- or 1,4-relative to each other, and when A is a five-membered ring or bicyclic heterocyclyl group the R¹ and cyclopentene group are attached to substitutable carbon atoms 1,2- or 1,3-relative to each other;
   or pharmaceutically acceptable derivatives thereof.
   When A is a six membered ring, preferably R¹ is attached to the group A in the 3 position relative to the bond attaching A to the cyclopentene ring. Preferably R¹ represents CO₂R⁴, wherein R⁴ is hydrogen or C₁₋₄alkyl.

Preferably A is selected from phenyl, pyridyl, pyridazinyl, pyrazinyl or pyrimidinyl, all of which may be optionally substituted. In an other aspect, A is selected from an optionally substituted phenyl, pyridyl, pyridazinyl, pyrazinyl or pyrimidinyl; more preferably A is pyridyl or an optionally substituted phenyl; most preferably A is optionally substituted phenyl. In an alternative aspect A is pyridyl.

In an alternative aspect:
A represents an optionally substituted phenyl, or an optionally substituted 5- or 6-membered heterocyclyl group;
R¹ represents CO₂R⁴, CONR⁵R⁶, CH₂CO₂R⁴, optionally substituted C₁₋₆alkyl, optionally substituted C₁₋₆alkenyl, SO₂C₁₋₆alkyl, SO₂NR⁵R⁶, NR⁵CONR⁵R⁶, tetrazolyl or CONR⁵R⁶;
R² independently represents halo, optionally substituted C₁₋₆alkyl, CN, SO₂R⁵, SR⁵, NO₂, optionally substituted aryl, CONR⁵R⁶ or optionally substituted heteroaryl;
R^{x} represents optionally substituted C₁₋₈alkyl or optionally substituted —CH₂-phenyl;
R⁴ represents hydrogen or an optionally substituted C₁₋₆alkyl;
R⁵ represents hydrogen or an optionally substituted C₁₋₆alkyl;
R⁶ represents hydrogen or an optionally substituted C₁₋₆alkyl, optionally substituted SO₂aryl, optionally substituted SO₂heterocyclyl group, CN, optionally substituted CH₂aryl or COR⁷;
R⁷ represents hydrogen or an optionally substituted aryl;
R⁸ and R⁹ independently represent hydrogen or C₁₋₆alkyl;
n is an integer from 0 to 2;
wherein R¹ is attached to the group A in the 3 or 4 position relative to the bond attaching A to the cyclopentene ring;
   or pharmaceutically acceptable derivatives thereof.

In a further aspect, A is optionally substituted phenyl or a 5 or 6-membered heterocyclyl group.

Optional substituents for A when a phenyl group include up to four substituents, preferably 0 or 1 substituent, independently selected from halogen, NR⁵R⁶, NR⁵COC₁₋₆alkyl, NR⁵SO₂C₁₋₆alkyl, OR⁵, C₁₋₆alkyl and NR¹⁰R¹¹ wherein R¹⁰ and R¹¹ together with the nitrogen atom to which they are attached form a morpholine ring, a 5- or 6-membered lactam ring or a 5- or 6-membered cyclic sulphonamide, wherein R⁵ and R⁶ are as defined above. Preferably optional substituents for A are selected from halogen, NR⁵R⁶, NHCOC₁₋₆alkyl, NHSO₂C₁₋₆alkyl, C₁₋₆alkyl and NR¹⁰R¹¹.

In an alternative aspect optional substituents for A when a phenyl group include up to four substituents independently selected from C₁₋₆alkyl, C₁₋₆alkoxy and halogen. Preferably A when a phenyl group is optionally substituted by up to 2 substiiuents.

Optional substituents for A when a 5- or 6-membered heterocyclyl group include NH₂. When A is pyridyl it may be substitued on the ring nitrogen by an oxygen to give a pyridine N-oxide. In an alternative aspect R¹ represents CO²R⁴, CONR⁵R⁶, CH₂CO₂R⁴, optionally substituted C₁₋₆alkyl, optionally substituted C₁₋₆alkenyl, SO₂C₁₋₆alkyl, SO₂NR⁵R⁶, NR⁵CONR⁵R⁶, tetrazolyl or COSO₂NR⁵R⁶.

In another aspect R² independently represents halo, optionally substituted C₁₋₆alkyl, CN, SO₂R⁵, NO₂, optionally substituted aryl, CONR⁵R⁶ or optionally substituted heteroaryl. In an alternative aspect R⁶ represents hydrogen or an optionally substituted C₁₋₆alkyl, optionally substituted SO₂aryl, optionally substituted SO₂heterocyclyl group, CN, or COR⁷.

Preferably R¹ represents CO₂R⁴. More preferably R¹ represents CO₂H.

Preferably R² represents halo, optionally substituted C₁₋₆alkyl e.g. C₁₋₄alkyl and CF₃, CN, SC₁₋₆alkyl, e.g SCH₃ or SO₂C₁₋₆alkyl, e.g. SO₂CH₃. Alternatively R² represents halogen, optionally substituted C₁₋₆alkyl, for example CF₃, CN or SO₂C₁₋₆alkyl.

Preferably R⁴ represents hydrogen or C₁₋₃alkyl.

Preferably R⁵ represents hydrogen or C₁₋₃alkyl.

Preferably R⁶ represents hydrogen or C₁₋₃alkyl.

Preferably R⁸ represents methyl or hydrogen, more preferably R⁸ represents hydrogen.

Preferably R⁹ represents hydrogen.

Preferably n is 0 or 1.

When R^{x} represents an optionally substituted alkyl this group is preferably C₁₋₈alkyl, more preferably the alkyl group is CH₂C₅₋₅cycloalkyl.

R^{x} preferably represents CH₂phenyl optionally substituted by one, two or three, preferably one or two substituents selected from Cl, Br, F, CF₃, C₁₋₄alkyl and OC₁₋₄alkyl or R^{x} is CH₂C₅₋₆cycloalkyl.

Preferred compounds of formula (I) are compounds of formula (II): 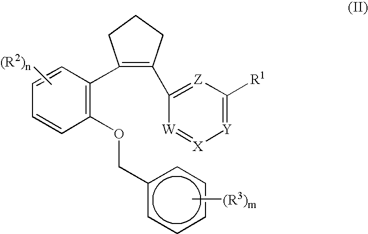
wherein:
R¹ is CO₂R⁴;
R² is halo, optionally substituted C₁₋₆alkyl e.g. C₁₋₄alkyl and CF₃, CN, SC₁₋₆alkyl, or SO₂C₁₋₆alkyl;
R³ independently represents halo, optionally substituted OC₁₋₆alkyl, or optionally substituted C₁₋₆alkyl;
m is an integer from 0 to 3;
n is an integer from 0 to 2; W, X, Y and Z each represents CR¹² or N wherein at least two of W, X, Y or Z is CR¹²; and when each of W, X, Y, and Z is CR¹² then each R¹² is independently selected from hydrogen, halogen, NR⁵R⁶, NHCOC₁₋₆alkyl, NHSO₂C₁₋₆alkyl, C₁₋₆alkyl and NR¹⁰R¹¹, and when at least one of W, X, Y and Z represents N then each R¹² is selected from hydrogen or NH₂;
   or pharmaceutically acceptable derivatives thereof.

In an alternative aspect of compounds of formula II:
R¹ is CO₂R⁴;
R² is halogen, optionally substituted C₁₋₆alkyl e.g. CF₃, CN, SC₁₋₆alkyl or SO₂C₁₋₆alkyl;
R³ independently represents halo or an optionally substituted OC₁₋₆alkyl, or C₁₋₆alkyl;
m is an integer from 0 to 2;
n is an integer from 0 to 2;
   W, X, Y and Z represents CH or N wherein at least one of W, X, Y or Z is CH;
   or pharmaceutically acceptable derivatives thereof.

In another aspect R² is halogen, optionally substituted C₁₋₆alkyl e.g. CF₃, CN, or SO₂C₁₋₆alkyl.

In a further aspect R³ represents halo, optionally substituted C₁₋₄alkyl e.g. CF₃, or optionally substituted OC₁₋₄alkyl, more preferably R³ is halo or OMe.

Preferred compounds include Examples 19, 29, 32, 52, 90, 140, and 153 in WO 03/084917, namely:
5-{2-[5-bromo-2-(2,4-difluorobenzyloxy)-phenyl]cyclopent-1-enyl}-nicotinic acid;
6-{2-[5-chloro-2-(2,4-difluorobenzyloxy)-phenyl]cyclopent-1-enyl}-pyridine-2-carboxylic acid;
6-{2-[5-chloro-2-(4-fluorobenzyloxy)-phenyl]-cyclopent-1-enyl}-pyridine 2 carboxylic acid;
6-{2-[5-trifluoromethyl-2-(benzyloxy)phenyl]cyclopent-1-enyl}-3-aminopyrazine-2-carboxylic acid;
6-{2-[5-trifluoromethyl-2-(benzyloxy)phenyl]cyclopent-1-enyl}pyrazine-2-carboxylic acid;
5-{2-[5-trifluoromethyl-2-(benzyloxy)phenyl]cyclopent-1-enyl}nicotinic acid;
6-{2-[5-chloro-2-(benzyloxy)phenyl]cyclopent-1-enyl}pyridine-2-carboxylic acid.

Further EP1 receptor antagonists that can be used in the present invention are compounds of formula III, as disclosed in WO 03/101959:

Accordingly the present invention provides the use of compounds of formula (III) for the treatment of LUTS associated with BPH: 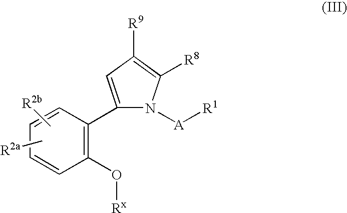
wherein:
A represents an optionally substituted aryl group, or an optionally substituted 5- or 6-membered heterocyclyl ring, or an optionally substituted bicyclic heterocyclyl group;
R¹ represents CO₂H, CN, CONR⁵R⁶, CH₂CO₂H, optionally substituted alkyl, optionally substituted alkenyl, optionally substituted SO₂alkyl, SO₂NR⁵R⁶, NR⁵CONR⁵R⁶, COalkyl, 2H-tetrazol-5-yl-methyl, optionally substituted bicyclic heterocycle or optionally substituted heterocyclyl;
R^{2a} and R^{2b} independently represents hydrogen, halo, optionally substituted alkyl, optionally substituted alkoxy, CN, SO₂alkyl, SR⁵, NO₂, optionally substituted aryl, CONR⁵R⁶ or optionally substituted heteroaryl;
R^{x} represents optionally substituted alkyl wherein 1 or 2 of the non-terminal carbon atoms may optionally be replaced by a group independently selected from NR⁴, O and SOₙ,
wherein n is 0, 1 or 2: or R^{x} may be optionally substituted CQ2-heterocyclyl, optionally substituted CQ2-bicyclic heterocyclyl or optionally substituted CQ₂-aryl;
R⁴ represents hydrogen or an optionally substituted alkyl;
R⁵ represents hydrogen or an optionally substituted alkyl;
R⁶ represents hydrogen or optionally substituted alkyl, optionally substituted heteroaryl, optionally substituted SO₂aryl, optionally substituted SO₂alkyl, optionally substituted SO₂heteroaryl, CN, optionally substituted CQ₂aryl, optionally substituted CQ₂heteroaryl or COR⁷;
R⁷ represents hydrogen, optionally substituted alkyl, optionally substituted heteroaryl or optionally substituted aryl;
R⁸ represents hydrogen, CF₃, or alkyl;
R⁹ represents hydrogen, CF₃ or alkyl;
Q is independently selected from hydrogen and CH₃;
wherein when A is a 6-membered ring the R¹ substituent and pyrrole ring are attached to carbon atoms 1,2-, 1,3- or 1,4-relative to each other, and when A is a five-membered ring or bicyclic heterocyclyl group the R¹ substituent and pyrrole ring are attached to substitutable carbon atoms 1,2- or 1,3-relative to each other;
   or a derivative thereof.

When A is a six membered ring, preferably the R¹ substituent is attached to A in the 3 or 4-position relative to the bond attaching A to the pyrrole ring. When R¹ is CO₂H, preferably the substituent is attached to A in the 3-position relative to the bond attaching A to the pyrrole ring.

Examples of A include phenyl, naphthyl, indolyl, pyridyl, pyridazinyl, pyrazinyl or pyrimidinyl, all of which may be optionally substituted. Particular examples include optionally substituted phenyl, optionally substituted pyridyl, indolyl or naphthyl.

Preferably A is pyridyl or an optionally substituted phenyl; most preferably A is optionally substituted phenyl. In an alternative embodiment A is preferably pyridyl, more preferably A is 2,6-disubstituted pyridyl. In an alternative aspect A is selected from phenyl, pyridyl, pyridazinyl, pyrazinyl and pyrimidinyl, all of which may be optionally substituted.

Examples of optional substituents for A when a phenyl group include up to four substituents, preferably up to three substituents, more preferably up to two substituents independently selected from halogen, C₁₋₄haloalkyl, C₁₋₄haloalkoxy, NR⁴R⁵, NR⁵COC₁₋₆alkyl, NR⁵SO₂C₁₋₆alkyl, OR⁵, C₁₋₆alkyl, SO₂C₁₋₆alkyl, NR⁵COCH₂OC₁₋₆alkyl, optionally substituted NR⁵COCH₂Oaryl, and optionally substituted NR⁵COCH₂heteroaryl, wherein R⁴ and R⁵ are each independently selected from hydrogen and C₁₋₄alkyl; and NR¹⁰R¹¹ wherein R¹⁰ and R¹¹ together with the nitrogen atom to which they are attached form an optionally substituted 5- or 6-membered aliphatic heterocyclic ring wherein one of the ring carbons may be optionally replaced by another heteroatom selected from O, and SOₙ wherein n is 0, 1 or 2.

Examples of substituents for the 5- or 6-membered aliphatic heterocyclic ring include oxo.

Preferably optional substituents for A when a phenyl group are selected from halogen, CF₃, OCHF₂, NR⁴R⁵, NR⁵COC₁₋₆alkyl, NR⁵SO₂C₁₋₆alkyl, OR⁵, C₁₋₆alkyl, SO₂C₁₋₆alkyl, NR₅COCH₂OC₁₋₆alkyl, NR⁵COCH₂thienyl, morpholinyl, pyrrolidinyl, 2-oxopyrrolidinyl, 2-oxopiperidinyl and 1,1-dioxo-1/⁶-isothiazolidinyl wherein R⁴ and R⁵ are each selected from hydrogen and C₁₋₄alkyl.

Optional substituents for A when a 5- or 6-membered heterocyclyl group include NH₂. When A is pyridyl it may be substituted on the ring nitrogen by an oxygen to give a pyridine N-oxide.

Examples of R¹ include CO₂H, CN, CONR⁴R⁵, optionally substituted CONR⁵SO₂aryl, optionally substituted CONR⁵SO₂heteroaryl, optionally substituted CONR⁵aryl, optionally substituted CONR⁵heteroaryl e.g. CONR⁵tetrazolyl and CONR⁵pyridyl, CONR⁵SO₂C₁₋₆alkyl, optionally substituted CONR⁵SO₂heteroaryl e.g.

CONR⁵SO₂-3,5-dimethylisoxazolyl, optionally substituted CONR⁵CQ₂aryl, optionally substituted CONR⁵CQ₂heteroaryl, optionally substituted C₁₋₆alkyl e.g. CF₃C(OH)CF₃, SO₂C₁₋₆alkyl, SO₂NR⁴R⁵, optionally substituted SO₂NR⁵COaryl, optionally substituted SO₂NR⁵COheteroaryl e.g SO₂NR⁵CO-3,5-dimethylisoxazolyl, SO₂NR⁵COC₁₋₆alkyl, optionally substituted SO₂NR⁵CQ₂aryl, optionally substituted SO₂NR⁵CQ₂heteroaryl; COC₁₋₆alkyl, 2H-tetrazol-5-yl-methyl, optionally substituted bicyclic heterocycyl e.g. benzimidazolyl, or optionally substituted heterocyclyl e.g. tetrazolyl, imidazolyl, methyloxadiazolyl and oxadiazolyl; wherein R⁴ and R⁵ are each selected from hydrogen and C₁₋₄alkyl, and Q is selected from hydrogen and CH₃.

When R¹ is optionally substituted heterocyclyl it is preferably tetrazolyl.

Preferably R¹ represents CONHCQ₂aryl, CONHCQ₂heteroaryl, CONHSO₂aryl, CONHSO₂heteroaryl, SO₂NHCOaryl, SO₂NHC Oheteroaryl all of which may be optionally substituted, CO₂H, tetrazolyl or SO₂CH₃. More preferably R¹ represents CONHCHQphenyl, CONHSO₂phenyl, SO₂NHCOphenyl, all of which may be optionally substituted, CO₂H, tetrazolyl or SO₂CH₃. Most preferably R¹ represents CO₂H.

Preferably aryl is optionally substituted phenyl.

Preferably Q is hydrogen.

When R^{x} represents an optionally substituted alkyl this group is preferably C₁₋₈alkyl, more preferably the alkyl group is CH₂C₅₋₆cycloalkyl wherein 1 or 2 of the ring carbon atoms may optionally be replaced by a group independently selected from NR⁴, O or SOₙ, wherein n is

0, 1 or 2 and R⁴ is selected from hydrogen and C₁₋₄alkyl.

Examples of R^{x} include CH₂CH(CH₃)₂, CH₂cyclohexyl, CH₂tetrahydrofuranyl, CH₂ tetrahydropyranyl, optionally substituted CH₂-heterocyclyl e.g. CH₂methylisoxazolyl, optionally substituted CH₂-bicyclic heterocyclyl e.g. CH₂benzofurazanyl, optionally substituted CH₂naphthyl or optionally substituted CH₂-phenyl. Examples of substituents for CH₂phenyl and CH₂naphthyl include up to 4 substituents independently selected from halogen, optionally substituted C₁₋₄alkyl, C₁₋₄haloalkyl, C₁₋₆haloalkoxy, optionally substituted phenyl, and optionally substituted OC₁₋₆alkyl. Particular examples include up to to three substituents independently selected from halogen, C₁₋₄alkyl, CF₃, phenyl, OC₁₋₄alkyl and OCHF₂. Preferred substituents include up to three substituents independently selected from chloro, bromo and fluoro.

In a preferred aspect R^{x} is optionally substituted CH₂-phenyl.

Preferably R^{2b} represents hydrogen, fluoro, chloro, bromo, optionally substituted C₁₋₄alkyl, e.g. CF₃, and CH₃, phenyl or SO₂C₁₋₄alkyl, e.g. SO₂CH₃. More preferably R^{2b} represents hydrogen, fluoro, chloro, bromo, or CF₃.

Preferably R^{2b} is positioned on the phenyl ring meta to the pyrrole group and para to the oxy substituent.

R⁴ is preferably hydrogen or C₁₋₆alkyl, more preferably hydrogen or C₄alkyl.

R⁵ is preferably hydrogen or C₁₋₆alkyl, more preferably hydrogen or C₁₋₄alkyl.

R⁸ preferably represents CH₃.

R⁹ preferably represents hydrogen.

In an alternative aspect:
A represents an optionally substituted phenyl, or a 5- or 6-membered heterocyclyl group;
R¹ represents CO₂R⁴, CONR⁵R⁶, CH₂CO₂R⁴, optionally substituted C₁₋₆alkyl, optionally substituted C₁₋₆alkenyl, SO₂C₁₋₆alkyl, SO₂NR⁵R⁶, NR⁵CONR⁵R⁶, tetrazolyl or CONR⁵R⁶;
R^{2a} and R^{2b} independently represent hydrogen, halo, CF₃, optionally substituted C₁₋₆alkyl, CN, SO₂R⁵, NO₂, optionally substituted aryl, CONR⁵R⁶ or optionally substituted heteroaryl;
R^{x} represents optionally substituted C₁₋₈alkyl or optionally substituted CH₂phenyl;
R⁴ represents hydrogen or an optionally substituted C₁₋₆alkyl;
R⁵ represents hydrogen or an optionally substituted C₁₋₆alkyl;
R⁶ represents hydrogen or an optionally substituted C₁₋₆alkyl, optionally substituted —SO₂aryl, optionally substituted SO₂heterocyclyl group, CN or COR⁷;
R⁷ represents hydrogen or an optionally substituted aryl;
R⁸ represents hydrogen, CF₃ or C₁₋₆alkyl;
R⁹ represents hydrogen, Cl, Br, I, CF₃ or C₁₋₆alkyl;
wherein R¹ is attached to the group A in the 3 position relative to the bond attaching A to the pyrrole ring;
   or a pharmaceutically acceptable derivative thereof.

Preferred compounds of formula (III) are compounds of formula (IIIa): 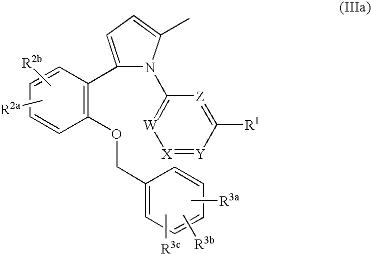
wherein:
R¹ is CO₂H;
R^{2a} and R^{2b} are independently selected from hydrogen, halo, phenyl, optionally substituted C₁₋₆alkyl e.g. C₁₋₄alkyl and CF₃, CN, SC₁₋₆alkyl, or SO₂C₁₋₆alkyl;
R^{3a}, R^{3b}, and R^{3c} are independently selected from hydrogen, halo, optionally substituted OC₁₋₆alkyl, e.g OCHF₂, phenyl or optionally substituted C₁₋₆alkyl e.g. CF₃;
W, X, Y and Z each represents CR¹² or N wherein at least two of W, X, Y or Z is CR¹²; and when each of W, X, Y, and Z is CR¹² then each R¹² is independently selected from hydrogen, halogen, C₁₋₄haloalkyl, C₁₋₄haloalkoxy, NR⁴R⁵, NR⁵COC₁₋₆alkyl, NR⁵SO₂C₁₋₆alkyl, OR⁵, C₁₋₆alkyl, SO₂C₁₋₆alkyl, NR⁵COCH₂OC₁₋₆alkyl, NR⁵OCH₂aryl, NR⁵COCH₂heteroaryl wherein R⁴ and R⁵ are each independently selected from hydrogen and C₁₋₄alkyl; and NR¹⁰R¹¹ wherein R¹⁰ and R¹¹ together with the nitrogen atom to which they are attached form an optionally substituted 5- or 6-membered aliphatic heterocyclic ring wherein one of the ring carbons may be optionally replaced by another heteroatom selected from O and SOₙ wherein n is 0, 1 or 2, and when at least one of W, X, Y and Z represents N then each R¹⁰ is selected from hydrogen and NH₂;
   or derivatives thereof.

In an alternative aspect of compounds of formula (IIIa):
R¹ is CO₂R⁴;
R^{2a} and R^{2b} are independently selected from hydrogen, halo, optionally substituted C₁₋₆alkyl, CN or SO₂C₁₋₆alkyl;
R^{3a} and R^{3b} are independently selected from hydrogen, halo or an optionally substituted OC₁₋₆alkyl, or C₁₋₆alkyl;
R^{3c} is hydrogen;
R⁴ is hydrogen or an optionally substituted C₁₋₆alkyl;
W, X, Y and Z represents CH or N wherein at least one of W, X, Y or Z is CH;
   or pharmaceutically acceptable derivatives thereof.

Preferably R^{2a} and R^{2b} are independently selected from hydrogen, chloro, fluoro, bromo and CF₃. More preferably R^{2a} is hydrogen and R^{2b} is selected from hydrogen, chloro, fluoro, bromo and CF₃.

Preferably R^{3a}, R^{3b} and R^{3c} are independently selected from hydrogen, CF₃, chloro, fluoro and bromo.

Preferably one of W, X, Y and Z is selected from N and CR¹² and the remaining atoms are CR². More preferably Z is N and W, X and Y are CR². Most preferably Z is N and W, X and Y are CH. Alternatively W, X, Y and Z are each selected from CR¹².

Preferred compounds of formula (III) in WO 03/101959 include:
3-{2-[5-bromo-2-(4-fluoro-benzyloxy)-phenyl]-5-methyl-pyrrol-1-yl}-benzoic acid;
5-{2-[5-chloro-2-(4-chloro-benzyloxy)-phenyl]-5-methyl-pyrrol-1-yl}-nicotinic acid;
3-{2-[5-chloro-2-(4-fluorobenzyloxy)-phenyl]-5-methylpyrrol-1-yl}-5-acetylamino-benzoic acid;
3-{2-[5-chloro-2-(4-fluorobenzyloxy)-phenyl]-5-methylpyrrol-1-yl}-6-methyl-benzoic acid;
3-{2-[chloro-2-2,4-difluorobenzyloxy)-phenyl]-5-methylpyrrol-1-yl}-6-methyl-benzoic acid;
3-{2-[5-chloro-2-(2,4-difluorobenzyloxy)phenyl]-5-methylpyrrol-1-yl}-6-chloro-benzoic acid;
3-{2-[5-bromo-2-(2,4-difluorobenzyloxy)-phenyl]-5-methylpyrrol-1-yl}-5-acetylamino-benzoic acid;
3-{2-[5-methanesulfonyl-2-(2,4-difluoro-benzyloxy)-phenyl]-5-methyl-pyrrol-1-yl}-benzoic acid;
3-{2-[5-trifluoromethyl-2-(2-chloro-4-fluoro-benzyloxy)-phenyl]-5-methyl-pyrrol-1-yl}-benzoic acid;
3-[2-(5-chloro-2-benzyloxy-phenyl)-5-methyl-pyrrol-1-yl]-N-(1-phenylsulfonyl)-benzamide;
3-[2-(2-benzyloxy-phenyl)-5-methyl-pyrrol-1-yl]-N-(3,5-dimethyl-isoxazole-4-sulfonyl)-benzamide;
4-{2-[5-chloro-2-(2,4-difluoro-benzyloxy)-phenyl]-5-methyl-pyrrol-1-yl}-N-(1-phenyl-methanoyl)-benzenesulfonamide;
3-{2-[5-chloro-2-(benzyloxy)-phenyl]-5-methyl-pyrrol-1-yl}-N-[(S)-1-phenyl-ethyl]-benzamide;
3-{2-[5-bromo-2-(2,4-difluoro-benzyloxy)-phenyl]-5-methyl-pyrrol-1-yl}-5-acetylamino-N-[(S)-1-phenyl-ethyl]-benzamide;
4-{2-[5-bromo-2-(2,4-difluoro-benzyloxy-phenyl]-5-methyl-pyrrol-1-yl}-N-pyridin-2-yl-benzamide;
2-{2-[5-chloro-2-(4-chloro-benzyloxy)-phenyl]-5-methyl-pyrrol-1-yl}-isonicotinic acid;
3-{2-[5-bromo-2-(2,6-difluoro-benzyloxy)-phenyl]-5-methyl-pyrrol-1-yl}-benzoic acid;
3-{2-[5-bromo-2-(2,4,6-trifluoro-benzyloxy)-phenyl]-5-methyl-pyrrol-1-yl}-benzoic acid;
3-{2-[5-bromo-2-(2-fluoro-benzyloxy)-phenyl]-5-methyl-pyrrol-1-yl}-benzoic acid;
3-{2-[5-bromo-2-(2,4-dichloro-benzyloxy)-phenyl]-5-methyl-pyrrol-1-yl}-benzoic acid;
3-{2-[5-bromo-2-(benzyloxy)-phenyl]-5-methyl-pyrrol-1-yl}-6-acetylamino-benzoic acid;
3-{2-[5-bromo-2-(benzyloxy)-phenyl]-5-methyl-pyrrol-1-yl}-5-(1,1-dioxo-1/⁶-isothiazolidin-2-yl)-benzoic acid;
3-{2-[5-bromo-2-(benzyloxy)-phenyl]-5-methyl-pyrrol-1-yl}-5-amino-6-methylbenzoic acid;
3-{2-[5-bromo-2-(benzyloxy)-phenyl]-5-methyl-pyrrol-1-yl}-6-methyl-benzoic acid;
3-{2-[5-bromo-2-(benzyloxy)-phenyl]-5-methyl-pyrrol-1-yl}-6-methoxy-benzoic acid;
3-{2-[5-bromo-2-(2,4,6-trifluoro-benzyloxy)-phenyl]-5-methyl-pyrrol-1-yl}-5-amino-6-methyl-benzoic acid;
3{-2-[5-bromo-2-(2,4,6-trifluoro-benzyloxy)-phenyl]-5-methyl-pyrrol-1-yl}-6-methyl-benzoic acid;
3-{2-[5-bromo-2-(4-fluoro-benzyloxy)-phenyl]-5-methyl-pyrrol-1-yl}-6-fluoro-benzoic acid;
3-{2-[5-bromo-2-(4-fluoro-benzyloxy)-phenyl]-5-methyl-pyrrol-1-yl}-6-hydroxy-benzoic acid;
3{2-[5-bromo-2-(4-fluoro-benzyloxy)-phenyl]-5-methyl-pyrrol-1-yl}-5-amino-6-methyl-benzoic acid;
3{2-[5-fluoro-2-(4-fluoro-benzyloxy)-phenyl]-5-methyl-pyrrol-1-yl}-6-methyl-benzoic acid;
3-{2-[5-fluoro-2-(4-fluoro-benzyloxy)-phenyl]-5-methyl-pyrrol-1-yl}-5-amino-6-methyl-benzoic acid;
3-{2-[5-fluoro-2-(2,4-difluoro-benzyloxy)-phenyl]-5-methyl-pyrrol-1-yl}-6-methyl-benzoic acid;
6-{2-[5-trifluoromethyl-2-(benzyloxy)-phenyl]-5-methyl-pyrrol-1-yl}-picolinic acid;
6-{2-[5-trifluoromethyl-2-(4-fluoro-benzyloxy)-phenyl]-5-methyl-pyrrol-1-yl}-picolinic acid;
6-{2-[5-trifluoromethyl-2-(2,6-difluoro-benzyloxy)-phenyl]-5-methyl-pyrrol-1-yl}-picolinic acid;
6-{2-[5-chloro-2-(4-fluoro-benzyloxy)-phenyl]-5-methyl-pyrrol-1-yl}-picolinic acid;
6-{2-[5-bromo-2-(4-fluoro-benzyloxy)-phenyl]-5-methyl-pyrrol-1-yl}-picolinic acid;
3-{2-[5-trifluoromethyl-2-(4-fluoro-benzyloxy)-phenyl]-5-methyl-pyrrol-1-yl}-6-methyl-benzoic acid;
3-{2-[5-trifluoromethyl-2-(4-fluoro-benzyloxy)-phenyl]-5-methyl-pyrrol-1-yl}-5-amino-6-methyl-benzoic acid;
3-{2-[5-trifluoromethyl-2-benzyloxy-phenyl]-5-pyrrol-1-yl}-5-amino-6-methyl-benzoic acid;
3-{2-[5-trifluoromethyl-2-(2,4-difluoro-benzyloxy)-phenyl]-5-methyl-pyrrol-1-yl)-5-(methanesulfonyl)-benzoic acid;
4-{2-[5-trifluoromethyl-2-(2,4-difluoro-benzyloxy)-phenyl]-5-methyl-pyrrol-1-yl}-2-methyl-benzoic acid;
3-{2-[5-chloro-2-(2,6-difluoro-benzyloxy)-phenyl]-5-methyl-pyrrol-1-yl}-6-chloro-benzoic acid;
3-{2-[5-chloro-2-(2,6-difluoro-benzyloxy)-phenyl]-5-methyl-pyrrol-1-yl}-6-methyl-benzoic acid;
3-{2-[5-chloro-2-(2,3-difluoro-benzyloxy)-phenyl]-5-methyl-pyrrol-1-yl}-6-methyl-benzoic acid;
3-{2-[5-bromo-2-(2,4-difluoro-benzyloxy)-phenyl]-5-methyl-pyrrol-1-yl}-5-methanesulfonylamino-benzoic acid;
3-{2-[5-bromo-2-(2,4-difluoro-benzyloxy))-phenyl]-5-methyl-pyrrol-1-yl}-6-methoxy-benzoic acid;
3-{2-[5-bromo-2-(2,4-difluoro-benzyloxy)-phenyl]-5-methyl-pyrrol-1-yl}-5-(1,1-dioxo-1/⁶-isothiazolidin-2-yl}-benzoic acid;
3-{2-[5-methyl-2-(4-fluoro-benzyloxy)-phenyl]-5-methyl-pyrrol-1-yl}-6-difluoromethoxy-benzoic acid;
3-{2-[5-methyl-2-(4-fluoro-benzyloxy)-phenyl]-5-methyl-pyrrol-1-yl}-5-amino-6-methyl-benzoic acid;
3-{2-[5-chloro-2-(benzyloxy)-phenyl]-5-methyl-pyrrol-1-yl}-5-amino-6-methyl-benzoic acid;
3-{2-[5-chloro-2-(benzyloxy)-phenyl]-5-methyl-pyrrol-1-yl}-6-methyl-benzoic acid;
6-{2-[5-chloro-2-(benzyloxy)-phenyl]-5-methyl-pyrrol-1-yl}-1H-indole-4-carboxylic acid;
3-{2-[5-chloro-2-(benzyloxy)-phenyl]-5-methyl-pyrrol-1-yl}-5-methoxycarbonylamino-benzoic acid;
4-{2-[5-bromo-2-(benzyloxy)-phenyl]-5-methyl-pyrrol-1-yl}-N-(1-phenyl-methanoyl)-benzenesulfonamide;
4-{2-[5-chloro-2-(benzoxy)-phenyl]-5-methyl-pyrrol-1-yl}-N-(pyridin-2-ylmethyl)-benzamide;
3-{2-[5-bromo-2-(2,4-difluoro-benzyloxy)-phenyl]-5-methyl-pyrrol-1-yl}-6-methyl-benzoic acid;
3-{2-[5-bromo-2-(2,4-difluoro-benzyloxy)-phenyl)]-5-methypurrol-1-yl}-6-difluoromethoxy-benzoic acid;
5-(3-{2-[5-bromo-2-(2,4-difluoro-benzyloxy)-phenyl]-5-methyl-pyrrol-1-yl}-phenyl)-1H-tetrazole;
2-(4-{2-[5-bromo-2-(2,4-difluoro-benzyloxy)-phenyl]-5-methyl-pyrrol-1-yl}-phenyl)-1,1,1,3,3,3-hexafluoro-propan-2-ol;
5-(4-{2-[5-bromo-2-(2,4,6-trifluoro-benzyloxy)-phenyl]-5-methyl-pyrrol-1-yl}-benzyl)-1H-tetrazole;
4-{2-[5-chloro-2-(benzyloxy)-phenyl]-5-methyl-pyrrol-1-yl}-benzoic acid;
   and pharmaceutically acceptable derivatives thereof.

Suitable EP1 receptor antagonists also include compounds included in patent application WO 02/15902, preferably the compounds exemplified in WO 02/15902, even more preferably compound no 9 in WO 02/15902, preferably a pharmaceutically acceptable salt or solvate thereof. These are compounds of formula (A), disclosed in WO 01/19814 and WO 02/15902: 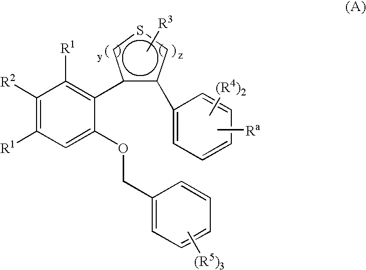
as well as pharmaceutically acceptable salts, hydrates and esters thereof, wherein:
y and z are independently 0-2, such that y+z=2;
R^{a} is selected from the group consisting of heteroaryl, wherein heteroaryl is selected from the group consisting of furyl, diazinyl, triazinyl or tetrazinyl, imidazolyl, isoxazolyl, isothiazolyl, oxadiazolyl, oxazolyl, pyrazolyl, pyrrolyl, thiadiazolyl, thiazolyl thienyl, triazolyl and tetrazolyl, said heteroaryl group being optionally substituted with one to three substituents selected from R¹¹ and C₁₋₄alkyl; —COR⁶; —NR⁷R¹¹; —SO₂R⁹; hydroxy; C₁₋₆alkoxy, optionally substituted with one to three substituents selected from R¹¹; and C₁₋₆alkyl, C₂₋₆alkenyl or C₃₋₆cycloalkyl, optionally substituted with one to three substituents selected from R¹¹, and further substituted with 1-3 substituents selected from the group consisting of —COR⁶; —NR⁷R⁸; —SO₂R⁹; hydroxy; C₁₋₆alkoxy or haloC₁₋₆alkoxy, and heteroaryl, such that R^{a} is positioned on the phenyl ring to which it is bonded in a 1, 3 or 1,4 relationship relative to the thienyl group represented in formula (A);
Each R¹, R², R³, R⁴ and R⁵ are independently selected from the group consisting of hydrogen, halogen, C₁₋₆alkyl, C₁₋₆alkoxy, C₁₋₆alkylthio, nitro, carboxy and CN, wherein C₁₋₆alkyl, C₁₋₆alkoxy, C₁₋₆alkylthio are optionally substituted with one or more substituents independently selected from R¹¹;
R⁶ is selected from the group consisting of hydrogen, hydroxy, C₁₋₆alkyl, C₁₋₆alkoxy and NR⁷R⁸, wherein C₁₋₆alkyl or C₁₋₆alkoxy are optionally substituted with one or more substituents independently selected from R¹¹;
R⁷ and R⁸ are independently selected from the group consisting of hydrogen, hydroxy, SO₂R⁹, C₁₋₆alkyl, C₁₋₆alkoxy, phenyl naphthyl, furyl, thienyl and pyridyl, wherein C₁₋₆alkyl and C₁₋₆alkoxy are optionally substituted with one or more substituents independently selected from R¹¹ or C₁₋₄alkyl;
R⁹ is selected from the group consisting of hydroxy, N(R¹⁰)₂, C₁₋₆alkyl, optionally substituted with one or more substituents independently selected from R¹¹, phenyl, naphthyl, furyl, thienyl and pyridyl, wherein phenyl, naphthyl, furyl, thienyl and pyridyl are optionally substituted with one or more substituents independently selected from R¹¹ or C₁₋₄alkyl;
R¹⁰ is hydrogen or C₁₋₆alkyl; and R¹¹ is the group consisting of halogen, hydroxy, C₁₋₃alkoxy, nitro, N(R¹⁰)₂ and pyridyl.

Of particular interest is the use of compounds of formula (A) for the treatment of LUTS associated with BPH wherein R^{a} is selected from the group consisting of heteroaryl as originally defined, COR⁶ wherein R⁶ is as originally defined, C₁₋₆ alkyl and C₂₋₆ alkenyl, optionally substituted as originally defined, and SO₂R⁹ with R⁹ as originally defined; all other variables are as originally defined.

Preferred is the use of the compounds exemplified in WO 01/19814, in particular the use of Example 9 (3-{3-[5-chloro-2-(phenylmethoxy)phenyl]2-thienyl}benzoic acid):

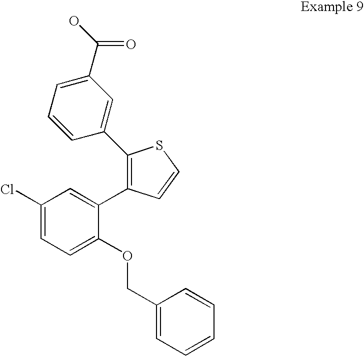

Yet a further aspect of the invention is a method of screening for compounds useful for treating the lower urinary tract symptoms (LUTS) associated with BPH, comprising screening compounds for antagonist activity against EP1 receptors, and selecting compounds with an IC₅₀ of less than 100 nM, preferably with an IC₅₀ of less than 10 nM, even more preferably with an IC₅₀ of less than 1 nM.

Another aspect of the invention is a process for providing a medicament for the treatment of the lower urinary tract symptoms (LUTS) associated with BPH, comprising the following steps:
(a) testing compounds in a ligand binding assay against EP1 receptors;
(b) selecting a compound with an IC₅₀ of less than 100 nM;
(c) formulating a compound with the same structure as that selected in step (b), or a pharmaceutically acceptable salt thereof, with a pharmaceutically acceptable carrier or excipient; the process may also comprise the additional steps of:
(d) packaging the formulation of step (c); and
(e) making the package of step (d) available to a patient suffering from the lower urinary tract symptoms (LUTS) associated with BPH.
   Preferably, the compound selected in step (b) will have an IC₅₀ of less than 10 nM, even more preferably it will have an IC₅₀ of less than 1 nM.

Yet another aspect of the invention is a process for providing a medicament for the treatment of the lower urinary tract symptoms (LUTS) associated with BPH, comprising the following steps:
(a) testing compounds in an assay, measuring the inhibition of the agonist-stimulated second messenger response of EP1 receptors;
(b) selecting a compound with an IC₅₀ of less than 100 nM;
(c) formulating a compound with the same structure as that selected in step (b), or a pharmaceutically acceptable carrier or excipient; the process may also comprise the additional steps of:
(d) packaging the formulation of step (c); and
(e) making the package of step (d) available to a patient suffering from the lower urinary tract symptoms (LUTS) associated with BPH.
   Preferably, the assay in step (a) measures a transient rise in intracellular calcium in EP1 receptor-expressing cells in response to an EP1 receptor agonist such as PGE₂, even more preferably, the transient rise in intracellular calcium is measured by fluorescence techniques, using calcium-sensitive fluorescent dyes such as Fluo-3. Preferably, the compound selected in step (b) will have an IC₅₀ of less than 10 nM, even more preferably it will have an IC₅₀ of less than 1 nM.

Another aspect of the invention is a process for preparing a medicament for the treatment of the lower urinary tract symptoms (LUTS) associated with BPH, comprising the steps of (a) testing compounds in a ligand binding assay against EP1 receptors or testing compounds in an assay, measuring inhibition of the agonist stimulated second messenger response of EP1 receptors; (b) identifying one or more compounds capable of antagonising EP1 receptors with an IC₅₀ of less than 100 nM; and (c) preparing a quantity of those one or more identified compounds. Preferably, the compound(s) selected in step (b) will have an IC₅₀ of less than 10 nM, even more preferably it/they will have an IC₅₀ of less than 1 nM.

Another aspect of the invention is a method of preparing a composition for treating the lower urinary tract symptoms (LUTS) associated with BPH which comprises:
(a) identifying a compound which specifically binds to EP1 receptors by a method which comprises contacting cells expressing EP1 receptors or membranes prepared from such cells with a radiolabelled EP1 receptor ligand (such as ³H-PGE₂) in the presence or absence of a test compound, measuring the radioactivity bound to the cells or membranes, comparing the radioactivity bound to the cells or membranes in the presence and absence of test compound, whereby a compound which causes a reduction in the radioactivity bound is a compound specifically binding to EP1 receptors; and
(b) admixing said compound with a carrier.

Yet another aspect of the invention is a method of preparing a composition for treating the lower urinary tract symptoms (LUTS) associated with BPH which comprises:
(a) identifying a compound which specifically binds to and inhibits the activation of EP1 receptors by a method which comprises separately contacting cells expressing EP1 receptors on their surface and producing a second messenger response in response to EP1 receptor agonist, e.g. PGE₂, or a membrane preparation of such cells, with both the compound and an agonist of EP1 receptors, and with only the agonist, under conditions suitable for activation of EP1 receptors, and measuring the second messenger response in the presence of only the agonist for EP1 receptors and in the presence of the agonist and the compound, a smaller change in the second messenger response in the presence of both agonist and compound than in the presence of the agonist only indicating that the compound inhibits the activation of EP1 receptors; and
(b) admixing said compound with a carrier.

Yet another aspect of the invention is a method of preparing a composition for treating the lower urinary tract symptoms (LUTS) associated with BPH which comprises:
(a) identifying a compound which specifically binds to and inhibits the activation of EP1 receptors by a method which comprises separately contacting cells expressing EP1 receptors on their surface and producing activation of a reporter gene such as beta-galactosidase or luciferase which in turn leads to a change in a measurable endpoint e.g. fluorescence or emitted light, in response to an EP1 receptor agonist, e.g. PGE₂, or a membrane preparation of such cells, with both the compound and an agonist of EP1 receptors, and with only the agonist, under conditions suitable for activation of EP1 receptors, and measuring the second messenger response in the presence of only the agonist for EP1 receptors and in the presence of the agonist and the compound, a smaller change in the second messenger response in the presence of both agonist and compound than in the presence of the agonist only indicating that the compound inhibits the activation of EP1 receptors; and
(b) admixing said compound with a carrier.

The invention relates to the use of an EP1 receptor antagonist for the treatment of the lower urinary tract symptoms (LUTS) associated with BPH alone, or in combination with one or more other agents such as α1 adrenergic receptor antagonists.

Reference to an antagonist, an agonist or an inhibitor shall at all times be understood to include all active forms of such agents, including the free form thereof (e.g. the free and/or base form) and also all pharmaceutically acceptable salts, polymorphs, hydrates, silicates, stereo-isomers (e.g. diastereoisomers and enantiomers) and so forth. Active metabolites of any of the compounds, in any form, are also included.

Particular formulations of the compounds for oral delivery or for topical application (creams, gels) or for intravesical administration are included in the invention.

Human EP1 receptor was cloned by Funk et al ((1993) J. Biol. Chem. 268, 26767-26772), and the sequence was deposited in GenBank/EMBL with Accession number L22647. The mouse EP1 receptor was cloned by Watanabe et al ((1993) J. Biol. Chem. 268, 20175-20178), and the sequence was deposited in GenBank/EMBL with Accession number D16338. The reference to EP1 receptors includes genetic variants thereof, e.g. as disclosed in patent applications WO 00/29614 or EP 1130122, as well as homologues and other variants.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “protein”.

In the present context, a homologous sequence is taken to include an amino acid sequence which may be at least 75, 85 or 90% identical to the amino acid sequence of the human EP1 receptor shown in Funk et al ((1993) J. Biol. Chem. 268, 26767-26772), preferably at least 95 or 98% identical. In particular, homology should typically be considered with respect to those regions of the sequence known to be essential for an activity. Although homology can also be considered in terms of similarity (i.e. amino acid residues having similar chemical properties/functions), in the context of the present invention it is preferred to express homology in terms of sequence identity. Such sequence homology/identity can be easily assessed by publicly or commercially available bioinformatics software, such as Blast2 (Altschul, S. F. et al (1997) Nucl. Acids Res. 25, 3389-3402), or programs included in the GCG software package (Devereux et al (1984) Nucl. Acids Res. 12, 387; Wisconsin Package Version 10, Genetics Computer Group (GCG, Madison, Wis.), such as Bestfit or Gap. In most cases, the default parameters offered by the software, e.g. Bestfit or Gap, for Gap Penalties etc. are suitable for this assessment.

“Potency” as used herein is a measure of the concentration of a compound at which it is effective. The potency of a compound can be determined in a binding assay as described in Example 2, and potency in this context will refer to the IC₅₀ of the compound, i.e. to the concentration inhibiting 50% of the labelled compound from binding to the receptors. The potency of a compound can also be determined in a functional assay such as cystometry measurements as described in Example 1. The potency in this case would refer to the IC₅₀ of the compound, i.e. the concentration which inhibits 50% of the functional response seen by application of the agonist.

“Selectivity” as used herein is a measure of the relative potency of a drug between two receptor subtypes for the same endogenous ligand. This can be determined in binding assays as described in Example 2, or in functional assays as described in Example 3, utilising cells or tissues expressing EP2, EP3 or EP4 receptors.

For the avoidance of doubt, the term “compound” may refer to a chemical or biological agent, and includes, for example, antibodies, antibody fragments, other proteins, peptides, sugars, any organic or inorganic molecules. Compounds that may be used for screening include, but are not limited to, peptides such as, for example, soluble peptides, including but not limited to members of random peptide libraries; (see, e.g., Lam et al. (1991) Nature 354, 82-84; Houghten et al. (1991) Nature 354, 84-86), and combinatorial chemistry-derived molecular library made of D- and/or L-configuration amino acids, phosphopeptides (including, but not limited to, members of random or partially degenerate, directed phosphopeptide libraries; see, e.g., Songyang et al. (1993) Cell 72, 767-778), antibodies (including, but not limited to, polyclonal, monoclonal, humanized, anti-idiotypic, chimeric or single chain antibodies, and Fab, F(ab′)₂ and Fab expression library fragments, and epitope-binding fragments thereof), and small organic or inorganic molecules.

The skilled person will be well aware how to obtain antibodies or antibody fragments that recognise EP1 receptors and can then be screened by the methods of the invention for their potential to be suitable for use in the treatment of the lower urinary tract symptoms (LUTS) associated with BPH. For the production of antibodies, various host animals may be immunized by injection with EP1 receptor, an EP1 receptor peptide (e.g. one corresponding to extracellular loops or the extracellular domain), truncated EP1 receptor polypeptides (EP1 receptor in which one or more domains, e.g. the transmembrane domain or cellular domain, has been deleted), functional equivalents of EP1 receptors or mutants of EP1 receptors. Such host animals may include but are not limited to rabbits, mice, hamsters and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Polyclonal antibodies are heterogeneous populations of antibody molecules derived from the sera of the immunized animals.

Monoclonal antibodies, which are homogeneous populations of antibodies to a particular antigen, may be obtained by any technique which provides for the production of antibody molecules by continuous cell lines in culture. These include, but are not limited to, the hybridoma technique of Kohler and Milstein, ((1975) Nature 256, 495-497 and U.S. Pat. No. 4,376,110), the human B-cell hybridoma technique (Kosbor et al. (1983) Immunology Today 4, 72; Cole et al. (1983) Proc. Natl. Acad. Sci. USA 80, 2026-2030), and the EBV-hybridoma technique (Cole et al. (1985) Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al. (1984) Proc. Natl. Acad. Sci., 81, 6851-6855; Neuberger et al. (1984) Nature, 312, 604-608; Takeda et al. (1985) Nature, 314, 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. No. 4,946,778; Bird (1988) Science 242, 423-426; Huston et al. (1988) Proc. Natl. Acad. Sci. USA 85, 5879-5883; and Ward et al. (1989) Nature 334, 544-546) can be adapted to produce single chain antibodies against EP1 receptor gene products. Single chain antibodies are formed by linking the heavy and light chain fragments of the Fv region via an amino acid bridge, resulting in a single chain polypeptide.

Antibody fragments which recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments or by papain digestion of antibody molecules. Alternatively, Fab expression libraries may be constructed (Huse et al. (1989) Science, 246, 1275-1281) to allow rapid and easy identification of monoclonal Fab fragments with the desired specificity.

Antibodies to EP1 receptors may also be obtained by generating anti-idiotype antibodies against the EP1 receptor ligand (prostaglandin E2), using techniques well known to those skilled in the art (see, e.g. Greenspan & Bona (1993) FASEB J 7, 437-444; and Nissinoff (1991) J. Immunol. 147, 2429-2438).

The suitability of the EP1 receptor antagonists can be readily determined by evaluation of their potency and selectivity using methods such as those disclosed herein, followed by evaluation of their toxicity, pharmacokinetics (absorption, metabolism, distribution and elimination), etc in accordance with standard pharmaceutical practice. Suitable compounds are those that are potent and selective, have no significant toxic effect at the therapeutic dose, and preferably are bioavailable following oral administration.

Oral bioavailablity refers to the proportion of an orally administered drug that reaches the systemic circulation. The factors that determine oral bioavailability of a drug are dissolution, membrane permeability and hepatic clearance. Typically, a screening cascade of firstly in vitro and then in vivo techniques is used to determine oral bioavailablity.

Dissolution, the solubilisation of the drug by the aqueous contents of the gastro-intestinal tract (GIT), can be predicted from in vitro solubility experiments conducted at appropriate pH to mimic the GIT. Preferably the EP1 receptor antagonists have a minimum solubility of 50 μg/ml. Solubility can be determined by standard procedures known in the art such as described in Lipinski C A et al.; Adv. Drug Deliv. Rev. 23(1-3), 3-25, 1997.

Membrane permeability refers to the passage of a compound through the cells of the GIT. Lipophilicity is a key property in predicting this and is determined by in vitro Log D₇₄ measurements using organic solvents and buffer. Preferably the EP1 receptor antagonists have a Log D_{7.4} of −2 to +4, more preferably −1 to +3. The Log D can be determined by standard procedures known in the art such as described in Stopher, D and McClean, S; J. Pharm. Pharmacol. 42(2), 144, 1990.

Cell monolayer assays such as Caco2 add substantially to prediction of favourable membrane permeability in the presence of efflux transporters such as P-glycoprotein, so-called Caco2 flux. Preferably, the EP1 receptor antagonists have a Caco2 flux of greater than 2×10⁻⁶ cms⁻¹, more preferably greater than 5×10⁻⁶ cms⁻¹. The Caco2 flux value can be determined by standard procedures known in the art such as described in Artursson, P and Magnusson, C; J. Pharm. Sci, 79(7), 595-600, 1990.

Metabolic stability addresses the ability of the GIT to metabolise compounds during the absorption process or the liver to do so immediately post-absorption: the first pass effect. Assay systems such as microsomes, hepatocytes etc are predictive of metabolic lability. Preferably EP1 receptor antagonists show metabolic stability in the assay system that is commensurate with an hepatic extraction of less then 0.5. Examples of assay systems and data manipulation are described in Obach, R S; Curr. Opin. Drug Disc. Devel. 4(1), 3644, 2001 and Shibata, Y et al.; Drug Met. Disp. 28(12), 1518-1523, 2000.

Because of the interplay of the above processes, further support that a drug will be orally bioavailable in humans can be gained by in vivo experiments in animals. Absolute bioavailability is determined in these studies by administering the compound separately or in mixtures by the oral route. For absolute determinations (% orally bioavailable) the intravenous route is also employed. Examples of the assessment of oral bioavailability in animals can be found in Ward, K W et al.; Drug Met. Disp. 29(1), 82-87, 2001; Berman, J et al.; J. Med. Chem. 40(6), 827-829, 1997 and Han K S and Lee, M G; Drug Met. Disp. 27(2), 221-226, 1999.

The compounds of the invention can be administered alone but will generally be administered in admixture with a suitable pharmaceutical excipient, diluent or carrier selected with regard to the intended route of administration and standard pharmaceutical practice.

For example, the compounds of the invention can be administered orally, buccally or sublingually in the form of tablets, capsules, multi-particulates, gels, films, ovules, elixirs, solutions or suspensions, which may contain flavouring or colouring agents, for immediate-, delayed-, modified-, sustained-, pulsed- or controlled-release applications. The compounds of the invention may also be administered as fast-dispersing or fast-dissolving dosage forms or in the form of a high energy dispersion or as coated particles. Suitable formulations may be in coated or uncoated form, as desired.

Such solid pharmaceutical compositions, for example, tablets, may contain excipients such as microcrystalline cellulose, lactose, sodium citrate, calcium carbonate, dibasic calcium phosphate, glycine and starch (preferably corn, potato or tapioca starch), disintegrants such as sodium starch glycollate, croscarmellose sodium and certain complex silicates, and granulation binders such as polyvinylpyrrolidone, hydroxypropylmethylcellulose (HPMC), hydroxypropylcellulose (H PC), sucrose, gelatin and acacia. Additionally, lubricating agents such as magnesium stearate, stearic acid, glyceryl behenate and talc may be included.

The following formulation examples are illustrative only and are not intended to limit the scope of the invention. Active ingredient means a compound of the invention.

Formulation 1:

A tablet is prepared using the following ingredients Active ingredient (50 mg) is blended with cellulose (microcrystalline), silicon dioxide, stearic acid (fumed) and the mixture is compressed to form tablets.

Formulation 2:

An intravenous formulation may be prepared by combining active ingredient (100 mg) with isotonic saline (1000 ml)

The tablets are manufactured by a standard process, for example, direct compression or a wet or dry granulation process. The tablet cores may be coated with appropriate overcoats.

Solid compositions of a similar type may also be employed as fillers in gelatin or HPMC capsules. Preferred excipients in this regard include lactose, starch, a cellulose, milk sugar or high molecular weight polyethylene glycols. For aqueous suspensions and/or elixirs, the EP1 receptor antagonists may be combined with various sweetening or flavouring agents, colouring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients may be present both within the dosage form i.e. within the matrix, and/or on the dosage form, i.e. upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e. where the drug substance is insoluble a fast dispersing dosage form can be prepared and where the drug substance is soluble a fast dissolving dosage form can be prepared.

The compounds of the invention can also be administered parenterally, for example, intracavernosaly, intravenously, intra-arterially, intraperitoneally, intrathecally, intraventricularly, intraurethrally, intrasternally, intracranially, intramuscularly or subcutaneously, or they may be administered by infusion or needleless injection techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The following dosage levels and other dosage levels herein are for the average human subject having a weight range of about 65 to 70 kg. The skilled person will readily be able to determine the dosage levels required for a subject whose weight falls outside this range, such as children and the elderly.

The dosage of the combination of the invention in such formulations will depend on its potency, but can be expected to be in the range of from 1 to 500 mg of the EP1 receptor antagonist for administration up to three times a day. A preferred dose is in the range 10 to 100 mg (e.g. 10, 25, 50 and 100 mg) of the EP1 receptor antagonist which can be administered once, twice or three times a day (preferably once). However the precise dose will be as determined by the prescribing physician and will depend on the age and weight of the subject and severity of the symptoms.

For oral and parenteral administration to human patients, the daily dosage level of a compound of the invention will usually be from to 5 to 500 mg/kg (in single or divided doses).

Thus tablets or capsules may contain from 5 mg to 250 mg (for example 10 to 100 mg) of the compound of the invention for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention. The skilled person will appreciate that the compounds of the invention may be taken as a single dose as needed or desired (i.e. prn). It is to be appreciated that all references herein to treatment include acute treatment (taken as required) and chronic treatment (longer term continuous treatment).

The compounds of the invention can also be administered intranasally or by inhalation and are conveniently delivered in the form of a dry powder inhaler or an aerosol spray presentation from a pressurised container, pump, spray, atomiser or nebuliser, with or without the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A [trade mark]) or 1,1,1,2,3,3,3-heptafluoropropane (HFA 227EA [trade mark]), carbon dioxide or other suitable gas. In the case of a pressurised aerosol, the dosage unit may be determined by providing a valve to deliver a metered amount. The pressurised container, pump, spray, atomiser or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the compounds of the invention and a suitable powder base such as lactose or starch.

Aerosol or dry powder formulations are preferably arranged so that each metered dose or “puff” contains from 1 μg to 50 mg of a compound of the invention for delivery to the patient. The overall daily dose with an aerosol will be in the range of from 1 g to 50 mg which may be administered in a single dose or, more usually, in divided doses throughout the day.

Alternatively, the compounds of the invention can be administered in the form of a suppository or pessary, or they may be applied topically in the form of a gel, hydrogel, lotion, solution, cream, ointment or dusting powder. The compounds of the invention may also be dermally or transdermally administered, for example, by the use of a skin patch, depot or subcutaneous injection. They may also be administered by the pulmonary or rectal routes.

For application topically to the skin, the compounds of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compounds of the invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in published international patent applications WO91/11172, WO94/02518 and WO98/55148.

Oral administration of the compounds of the invention is a preferred route, being the most convenient. In circumstances where the recipient suffers from a swallowing disorder or from impairment of drug absorption after oral administration, the drug may be administered parenterally, sublingually or buccally.

### EXAMPLES

The examples below are carried out using standard techniques, which are well-known and routinely used by those skilled in the art; the examples illustrate but do not limit the invention.

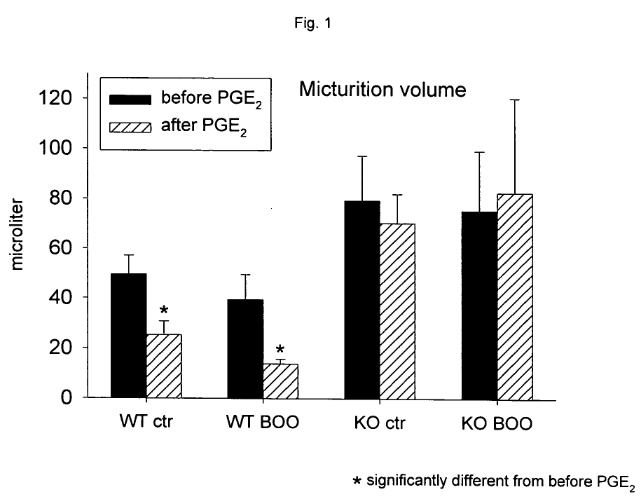

FIG. 1 shows a comparison of the micturition volume of wildtype and EP1 knockout mice with and without surgical bladder obstruction and the effects of intravesical PGE2 administration.

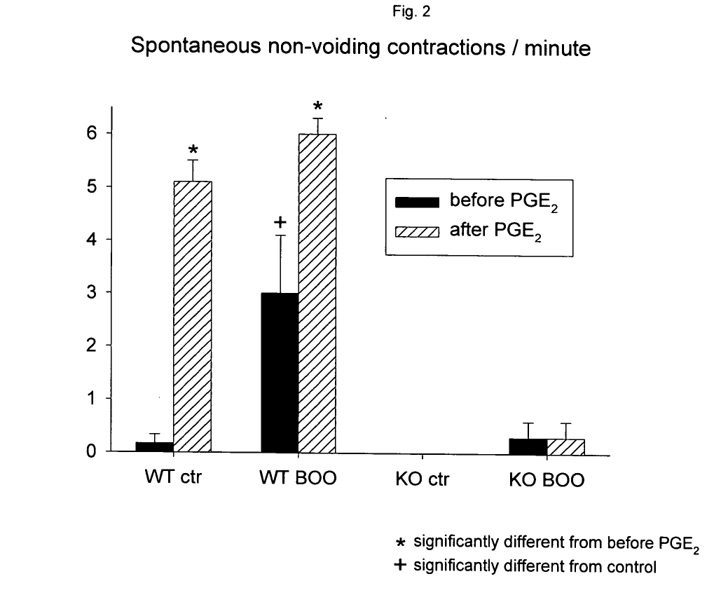

FIG. 2 shows the frequency of spontaneous non-voiding contractions in wildtype and EP1 knockout mice with and without surgical bladder obstruction. The effect of intravesical administration of PGE2 is also shown.

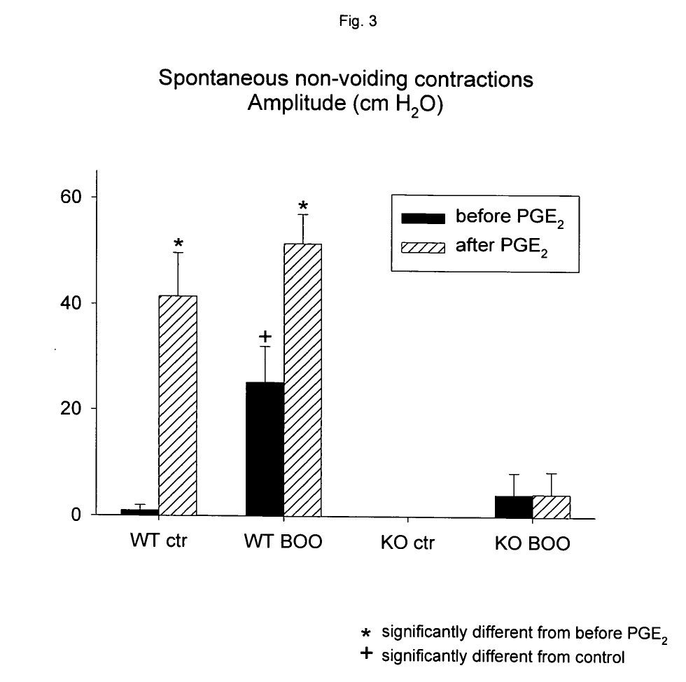

FIG. 3 shows the amplitude of spontaneous non-voiding contraction amplitudes in wildtype and EP1 knockout mice with and without surgical bladder obstruction. The effect of intravesical administration of PGE2 is also shown.

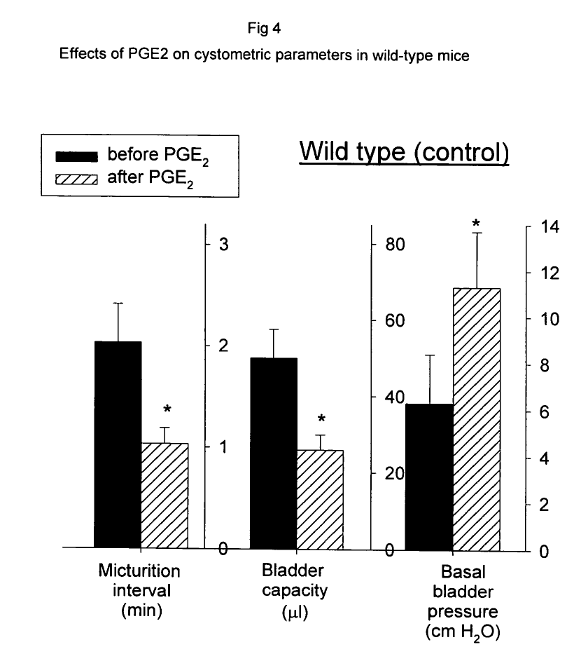

FIG. 4 shows the effects of PGE2 on cystometric parameters in wild-type mice.

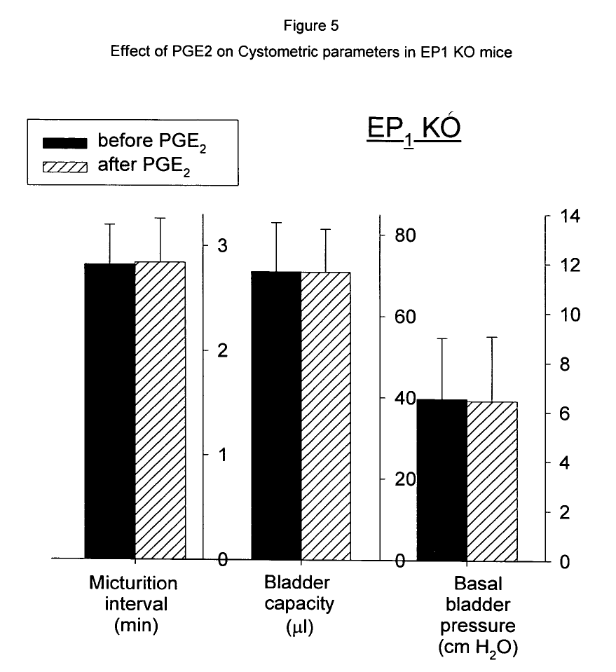

FIG. 5 shows the effects of PGE2 on cystometric parameters in EP1 Knockout mice.

### Example 1

### The Beneficial Effect of an EP1 receptor Antagonist in the Treatment of LUTS Associated with BPH, Using Transgenic EP1 Knockout Mice

Materials and Methods

Animals: Age-matched female EP1 knockout (EP1KO) mice (DBA/1LacJ background) (n=13) and DBA/1LacJ wild type (WT) controls (n=12) were used for the studies. The knockout mice originated from Groton Laboratories, Pfizer Research and Development, the wild type from the Jackson Laboratories, USA. Both strains were delivered through Charles River Laboratories, UK. After arrival, the mice were housed for 6 weeks under identical conditions under a 12 hours light/dark photocycle, food and water were provided ad libitum. The experimental protocol was approved by the Animals Ethics Committee, Lund University.

The knockout and WT mice were randomly divided into 3 groups each. One third received bladder outlet obstruction (BOO) as described below, one third received sham surgery. The remaining mice served as unoperated controls.

Surgical procedure: The mice in the BOO group were anesthetized with ketamine (Ketalar®, Parke Davis, Barcelona, Spain; 100 mg/kg IP) and xylazine (Rompun®, Bayer, Leverkusen, Germany, 15 mg/kg IP). The obstruction was created by a standardized method as described in Schroder et al 2003 J.Urol (in press). Sham operated animals received surgery similarly, without tying the obstruction.

At day 5 after the obstruction a polyethylene catheter (PE, ID 0.38 mm, OD 0.61 mm) with a small cuff was inserted in the bladder dome and secured with a purse-string suture (7-0 silk). The obstructing ligature remained in place. The catheter was tunneled subcutaneously, led out on the back of the neck, and surgically secured. Control animals received the bladder catheter 2 days prior to cystometry.

Cystometry: Two days after insertion of the catheter (7 days after creation of the obstruction), the cystometric investigation was performed without any anesthesia or restraint. The mice were placed into a metabolic cage (Gazzada, Buguggiatade, Italy). The bladder catheter was connected to a pressure transducer, which in turn was connected to a Grass® 7E Polygraph recorder. The bladder was continuously filled with saline at room temperature by means of a microinjecton pump (CMA 100, Carnegie Medicine, Solna, Sweden), at a filling speed of 25 μl/min.

The amount of voided urine was measured by means of a fluid collector, connected to a force displacement transducer (FT 03 D; Grass instrument Co., MA, USA). After a stabilization period of 60-80 minutes, in which the bladder was continuously filled, reproducible voiding patterns were achieved and recorded over a period of 30 minutes. The following parameters were measured: Micturition interval (time between 2 voids), baseline pressure (lowest pressure between 2 voids), threshold pressure (pressure immediately before micturition was initiated), micturition pressure (maximum voiding pressure), and micturition volume. Residual urine was emptied manually 3 times at the end of the cystometry and measured. Bladder capacity was calculated as the amount of saline infused into the bladder between 2 voids, plus the average amount of residual urine.

The animals were continuously observed in order to distinguish between moving artifacts and non-voiding bladder contractions. The surface of the collecting-funnel under the grid of the metabolic cage was sprayed with a thin layer of silicone.

Drugs and solutions: PGE₂ (Sigma Chemical Company, St. Louis, Mo., USA). PGE₂ was dissolved in ethanol, and the stock solution was stored at a concentration of 10⁻² M at −70°. The final dilution of 20 μM was made in NaCl at the day of cystometry. (Normal Krebs solution was composed as following (mM): NaCl 119, KCl 4.6, CaCl₂ 1.5, MgCl₂ 1.2, NaHCO₃ 15, NaH₂PO₄ 1.2, and glucose 11).

Data analysis: All values are reported as the mean±SEM. Statistical significance was determined by Student's t-test and Bonferroni correction as appropriate. P<0.05 was required for statistical significance.

Results:

Cystometry

WT Versus EP1KO Controls

No significant differences were found in the basic cystometry parameters (micturition interval and volume, micturition pressure, baseline pressure, and threshold pressure), comparing the control and sham operated WT and EP1KO mice, respectively. The control and sham groups of both strains were therefore merged and are hereafter called controls. Comparison of cystometry parameters between the WT and EP1KO control mice demonstrate that micturition interval and volume, and micturition pressure were larger in the knockout mice. Table 1 compares cystometrical parameters for wildtype and EP1 knockout mouse with and without bladder outlet obstruction.

BOO Versus Control

Following bladder outflow obstruction, WT mice developed frequent non-voiding contraction of high amplitude in between the voids (FIGS. 2 & 3, Table 1). In contrast, non-voiding contractions were rarely observed in the obstructed EP1KO mice and when present were of much lower amplitude than those in the WT mice (FIGS. 2 & 3, Table 1). Micturition pressure was significantly higher in the obstructed knockout mice, compared to obstructed WT mice. Furthermore, the WT mice showed a trend towards a decreased micturition interval and volume after BOO, and this was not seen in the EP1KO mice (FIG. 1, Table 1).

TABLE 1

| | WT | | | |
|---|---|---|---|---|
| | control | WT BOO | EP₁KO control | EP₁KO BOO |
| No. of ani- | 7 | 5 | 9 | 4 |
| mals | | | | |
| MI (minutes) | 2.0 ± 0.4 | 1.6 ± 0.3 | 2.8 ± 0.5 | 3.8 ± 1.4 |
| MV (μl) | 49.5 ± 7.6 | 39.3 ± 10.1 | 79.1 ± 18.0 | 75.1 ± 24.5 |
| BC (μl) | 50.9 ± 9.2 | 39.4 ± 6.5 | 70.9 ± 12.1 | 114.3 ± 54.8 |
| MP (cm | 36.0 ± 4.5 | 37.2 ± 2.2 | 41.3 ± 4.3 | 55.3 ± 5.3 |
| H₂O) | | | | |
| TP (cm | 12.5 ± 2.2 | 15.4 ± 2.1 | 12.6 ± 2.7 | 14.3 ± 1.4 |
| H₂O) | | | | |
| BP (cm | 6.3 ± 2.1 | 11.7 ± 2.1 | 6.5 ± 2.5 | 8.4 ± 0.7 |
| H₂O) | | | | |

Values are given as mean ± SEM.
MI = micturition interval;
MV = micturition volume;
BC = bladder capacity;
MP = micurition pressure;
TP = threshold pressure;
BP = basal pressure.

Effects of PGE₂

Under instillation of PGE₂, significant changes occurred in the WT mice. In the WT controls micturition interval, volume, and bladder capacity decreased (FIGS. 1, 4 and 5) Frequent non-voiding contractions of high amplitude occurred, which were negligible before PGE₂ (FIGS. 2 and 3). In the EP1 KO control animals, intravesical instillation of PGE₂ had no effect on micturition interval, micturition volume or bladder capacity (FIGS. 1 and 5). Furthermore, there was no induction of non-voiding contractions (FIGS. 2 and 3), in contrast to the WT mice (FIG. 3).

The data from the studies described above provide a number of novel observations regarding the role of the EP1 receptor in bladder function and in particular the altered bladder function which occurs following outlet obstruction. Mice which lack the EP1 receptor do not exhibit the changes in bladder function, particularly the appearance of non-voiding contractions, following bladder outlet obstruction which are observed in control animals. Thus it can be concluded that the EP1 receptor subtype plays a fundamental role in the appearance of non-voiding contractions and the other changes in bladder function following bladder outlet obstruction. These studies demonstrate the therapeutic potential of an agent which blocks signalling via the EP1 receptor for the treatment of bladder symptoms associated with BPH.

An additional novel observation in the studies described is that the bladder hyperactivity induced by intravesicular administration of PGE₂ is not present in EP1 KO mice and therefore that all of the direct actions of PGE₂ on the bladder are mediated via the EP1 receptor subtype.

Furthermore these data suggest that the changes in bladder function observed in BPH patients arising from bladder outflow obstruction associated with prostatic hypertrophy are also likely to involve signalling via the EP1 receptor and that antagonism or blocking of signalling via this receptor may well provide effective relief of the bladder symptoms associated with BPH.

### Example 2

### Ligand Binding Assay to Identify Antagonists for EP1 Receptors

Ligand binding assays can be carried out in native tissues expressing the EP1 receptor or using recombinant cell lines. The preferred method is to utilize stably expressing recombinant cell lines. EP1 binding affinity of test compounds is determined by their ability to displace [³H]-PGE₂ (Dupont NEN) from from cell membranes prepared from EP1 receptor expressing cells or tissues. Specific binding is determined using standard methodologies for filtration binding assays (e.g. as described by Kiriyama et al. (1997) Br.J. Pharmacol, 1997, 122, 217-224). Affinity Kᵢ values for test compounds are determined using IC₅₀ values detrmined from competition binding curves and K_{d} values measured for the ligand.

### Example 3

### Functional Assay (FLIPR)

Intracellular calcium release can be measured in CHO-EP1 cells using FLIPR, which allows the rapid detection of calcium following receptor activation. The CHO-EP1 cell line is maintained at 37° C. in humidified atmosphere with 5% CO₂ in DMEM/Hams F12 nutrient mix supplemented with 10% fetal bovine serum, 2 mM L-glutamine, 15 mM HEPES and 400 μg/ml G418. On the afternoon before the assay cells are plated at a density of 20,000 cells per well into black sterile 96-well plates with clear bottoms to allow cell inspection and fluorescence measurements from the bottom of each well. Wash buffer containing Dulbecco's phosphate buffered saline (DPBS) and 2.5 mM probenecid and loading dye consisting of cell culture medium containing 4 μM Fluo-3-AM (dissolved in DMSO and pluronic acid, Molecular Probes) and 2.5 mM probenecid is prepared fresh on the day of assay. Compounds are solubilised in DMSO and diluted in assay buffer consisting of DPBS containing 1% DMSO, 0.1% BSA and 2.5 mM probenecid. The cells are incubated with 100 μl loading dye per well for 1 hour at 37° C. in humidified atmosphere with 5% CO₂. After dye loading the cells are washed three times in 100 μl wash buffer using a Denley plate washer. 100 μl wash buffer is left in each well. Intracellular fluorescence is measured using FLIPR (Molecular Devices). Fluorescence readings are obtained at 2 s intervals with 50 μl of the test compound added after 30 s. An additional 155 measurements at 2 s intervals are then taken to detect any compound agonistic activity. 50 μl of prostaglandin E2 (PGE₂) is then added so that the final assay volume is 200 μl. Further fluorescence readings are collected at 1 s intervals for 120 s. Responses are measured as peak fluorescence intensity (FI). For pharmacological characterization a basal FI is subtracted from each fluorescence response. For PGE₂ dose response curves, each response is expressed as a % of the response to the highest concentration of PGE₂ in that row. For IC₅₀ determinations, each response is expressed as a % of the response to PGE₂. IC₅₀ values are converted to a modified Kb value using the Cheng-Prusoff equation which takes into account the agonist concentration, [A], the agonist EC₅₀ and the slope: K_{b}=IC₅₀(2+[A]/A₅₀]ⁿ)^{1/n}−1 where [A] is the concentration of PGE₂, A₅₀ is the EC₅₀ of PGE₂ from the dose response curve and n=slope of the PGE₂ dose response curve.

Another potential assay system for quantification of functional antagonist potency is the Alphascreen system (Perkin Elmer Life Sciences). This system detects increases in the second messenger IP3 in response to agonist activation of G protein coupled receptors based on competition between IP3 produced by the cell and a biotinylated IP3 analogue for binding to a GST tagged IP3 binding protein. EP1 antagonist potency against an agonist challenge can be determined using standard pharmacological techniques as described above for calcium fluorescence detection.

The skilled person will be able to adapt these assays for EP2, EP3 or EP4 receptors, using routine techniques.

### Example 4

### In Vivo Assay Demonstrating the Beneficial Effect of EP1 Receptor Antagonists for LUTS Associated with BPH

In vivo functional activity of an EP1 antagonist in normal animals is confirmed in animals with a functional EP1 receptor, using methodology very similar to the methodology described in Example 1. The assay can be performed in mouse, rat, marmoset or other suitable laboratory species.

An EP1 receptor antagonist with suitable affinity and selectivity is tested in standard pharmacokinetic tests to allow the development of a suitable dosing regime that will give suitable exposure to the compound in the animal. Depending on the compound used, a suitable route of administration is chosen, which is selected from intravenous, subcutaneous, intravesical, intra-peritoneal or oral routes. The skilled person uses standard techniques to select a suitable dosing regime, taking into account the pharmacokinetic profile and in vitro pharmacology profile of the compound.

Bladder activity is then tested as described in Example 1.

1. A method for treating lower urinary tract symptoms (LUTS) associated with BPH in a patient in need of such treatment which method comprises administering to said patient a therapeutically effective amount of an EP1 receptor antagonist

2. The method of claim 1, wherein the EP1 receptor antagonist is a compound of formula (A) 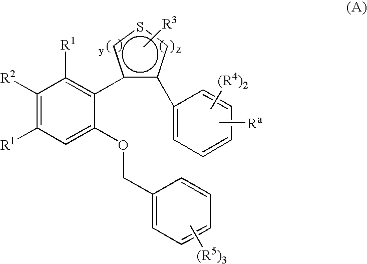
as well as pharmaceutically acceptable salts, hydrates and esters thereof, wherein:
y and z are independently 0-2, such that y+z=2;
R^{a} is selected from the group consisting of heteroaryl, wherein heteroaryl is selected from the group consisting of furyl, diazinyl, triazinyl or tetrazinyl, imidazolyl, isoxazolyl, isothiazolyl, oxadiazolyl, oxazolyl, pyrazolyl, pyrrolyl, thiadiazolyl, thiazolyl thienyl, triazolyl and tetrazolyl, said heteroaryl group being optionally substituted with one to three substituents selected from R¹¹ and C₁₋₄alkyl; —COR⁶; —NR⁷R⁸; —SO₂R⁹; hydroxy;
C₁₋₆alkoxy, optionally substituted with one to three substituents selected from R¹¹; and C₁₋₆alkyl, C₂₋₆alkenyl or C₃₋₆cycloalkyl, optionally substituted with one to three substituents selected from R¹¹, and further substituted with 1-3 substituents selected from the group consisting of —COR⁶; —NR⁷R⁸; —SO₂R⁹; hydroxy; C₁₋₆alkoxy or haloC₁₋₆alkoxy, and heteroaryl, such that R^{a} is positioned on the phenyl ring to which it is bonded in a 1, 3 or 1,4 relationship relative to the thienyl group represented in formula (A);
Each R¹, R², R³, R⁴ and R⁵ are independently selected from the group consisting of hydrogen, halogen, C₁₋₆alkyl, C₁₋₆alkoxy, C₁₋₆alkylthio, nitro, carboxy and CN, wherein
C₁₋₆alkyl, C₁₋₆alkoxy, C₁₋₆alkylthio are optionally substituted with one or more substituents independently selected from R¹¹;
R⁶ is selected from the group consisting of hydrogen, hydroxy, C₁₋₆alkyl, C₁₋₆alkoxy and NR⁷R⁸, wherein C₁₋₆alkyl or C₁₋₆alkoxy are optionally substituted with one or more substituents independently selected from R¹¹;
R⁷ and R⁸ are independently selected from the group consisting of hydrogen, hydroxy, SO₂R⁹, C₁₋₆alkyl, C₁₋₆alkoxy, phenyl naphthyl, furyl, thienyl and pyridyl, wherein C₁₋₆alkyl and C₁₋₆alkoxy are optionally substituted with one or more substituents independently selected from R¹¹ or C₁₋₄alkyl;
R⁹ is selected from the group consisting of hydroxy, N(R¹⁰)₂, C₁₋₆alkyl, optionally substituted with one or more substituents independently selected from R¹¹, phenyl, naphthyl, furyl, thienyl and pyridyl, wherein phenyl, naphthyl, furyl, thienyl and pyridyl are optionally substituted with one or more substituents independently selected from R¹¹ or C₁₋₄alkyl;
R¹⁰ is hydrogen or C₁₋₆alkyl; and
R¹¹ is the group consisting of halogen, hydroxy, C₁₋₃alkoxy, nitro, N(R¹⁰)₂ and pyridyl.

3. The method of claim 1, wherein the EP1 receptor antagonist is selected from ONO-8711, ONO-8713, 3-{3-[5-chloro-2-(phenylmethoxy)phenyl]2-thienyl}benzoic acid or SC-51089 in the manufacture of a medicament for the treatment of the lower urinary tract symptoms (LUTS) associated with BPH.

4. The method of claim 1, wherein the IC₅₀ of the antagonist for EP1 receptors is less than 100 nM.

5. The method of claim 1, wherein the antagonist for EP1 receptors is selective for EP1 receptors.

6. A method of screening for compounds useful for the treatment of the lower urinary tract symptoms (LUTS) associated with BPH, comprising screening compounds for antagonist activity against EP1 receptors, and selecting compounds with an IC₅₀ of less than 100 nM.

7. A process for providing a medicament for the treating lower urinary tract symptoms (LUTS) associated with BPH, which comprises the steps of:
(a) testing compounds in a ligand binding assay against EP1 receptors;
(b) selecting a compound with an IC₅₀ of less than 100 nM;
(c) formulating a compound with the same structure as that selected in step (b), or a pharmaceutically acceptable salt thereof, with a pharmaceutically acceptable carrier or excipient.

8. A process for providing a medicament for treating lower urinary tract symptoms (LUTS) associated with BPH, which method comprises the steps of:
(a) testing compounds in an assay, measuring the inhibition of the agonist-stimulated second messenger response in cells expressing EP1 receptors;
(b) selecting a compound with an IC₅₀ of less than 100 nM;
(c) formulating a compound with the same structure as that selected in step (b), or a pharmaceutically acceptable salt thereof, with a pharmaceutically acceptable carrier or excipient.

9. The process of claim 7 or claim 8, further comprising the steps of:
(d) packaging the formulation of step (c);
(e) making the package of step (d) available to a patient suffering from the lower urinary tract symptoms (LUTS) associated with BPH.

10. A process for preparing a medicament for treating lower urinary tract symptoms (LUTS) associated with BPH, which process comprises the steps of:
(a) testing compounds in a ligand binding assay against EP1 receptors or testing compounds in an assay, measuring the inhibition of the agonist-stimulated second messenger response of EP1 receptors,
(b) identifying one or more compounds capable of antagonising EP1 receptors with an IC₅₀ of less than 100 nM; and
(c) preparing a quantity of those one or more identified compounds.

11. A method of preparing a composition for treating lower urinary tract symptoms (LUTS) associated with BPH which method comprises the steps of:
(a) identifying a compound which specifically binds to EP1 receptors by a method which comprises contacting cells expressing EP1 receptors or membranes prepared from such cells with a radiolabelled EP1 receptor ligand in the presence or absence of a test compound, measuring the radioactivity bound to the cells or membranes in the presence and absence of test compound, whereby a compound which causes a reduction in the radioactivity bound is a compound specifically binding to EP1 receptors; and
(b) admixing said compound with a carrier.

12. A method of preparing a composition for treating lower urinary tract symptoms (LUTS) associated with BPH which method comprises the steps of:
(a) identifying a compound which specifically binds to and inhibits the activation of EP1 receptors by a method which comprises separately contacting cells expressing EP1 receptors on their surface and producing a second messenger response in response to an EP1 receptor agonist, or a membrane preparation of such cells, with both the compound and an agonist of EP1 receptors, and with only the agonist, under conditions suitable for activation of EP1 receptors, and measuring the second messenger response in the presence of only the agonist for EP1 receptors and in the presence of the agonist and the compound, a smaller change in the second messenger response in the presence of both agonist and compound than in the presence of the agonist only indicating that the compound inhibits the activation of EP1 receptors; and
(b) admixing said compound with a carrier.

13. A method of preparing a composition for treating lower urinary tract symptoms (LUTS) associated with BPH which method comprises the steps of:
(a) identifying a compound which specifically binds to and inhibits the activation of EP1 receptors by a method which comprises separately contacting cells expressing EP1 receptors on their surface and producing activation of a reporter gene such as beta-galactosidase or luciferase which in turn leads to a change in a measurable endpoint e.g. fluorescence or emitted light, in response to an EP1 receptor agonist, or a membrane preparation of such cells, with both the compound and an agonist of EP1 receptors, and with only the agonist, under conditions suitable for activation of EP1 receptors, and measuring the second messenger response in the presence of only the agonist for EP1 receptors and in the presence of the agonist and the compound, a smaller change in the second messenger response in the presence of both agonist and compound than in the presence of the agonist only indicating that the compound inhibits the activation of EP1 receptors; and
(b) admixing said compound with a carrier.
